(19) United States
(12) Patent Application Publication
(10) Pub. No.: US 2016/0296163 A1
(43) Pub. Date: Oct. 13, 2016

(54) METHOD, SYSTEM AND APPARATUS FOR A TOOTHBRUSH WITH SMART SENSORS FOR COMMUNICATION WITH ELECTRONIC DEVICES

(71) Applicants: Mubashir A. CHAUDHRY, North Miami Beach, FL (US); Aasim M. SAIED, North Miami Beach, FL (US)

(72) Inventors: Mubashir A. CHAUDHRY, North Miami Beach, FL (US); Aasim M. SAIED, North Miami Beach, FL (US)

(21) Appl. No.: 15/036,450

(22) PCT Filed: Jun. 3, 2014
(86) PCT No.: PCT/US14/40773

§ 371 Date: May 13, 2016

Related U.S. Application Data
(60) Provisional application No. 61/904,419, filed on Nov. 14, 2013.

(51) Int. Cl. A61B 5/00; A46B 15/00; A61C 17/22; G06F 21/62; A61B 5/145; A61B 5/01; A61B 5/03; H04W 4/00; A46B 9/04; A61B 5/08

(52) U.S. Cl. CPC A61B 5/486; A46B 9/04; A46B 15/001; A46B 15/004; A46B 15/0008; A61C 17/221; A61B 5/082; A61B 5/14507; A61B 5/4547; A61B 5/01; A61B 5/038; H04W 4/008; G06F 21/6245

(57) ABSTRACT

Method, system and apparatus for a toothbrush with smart sensors for communication with electronic devices.

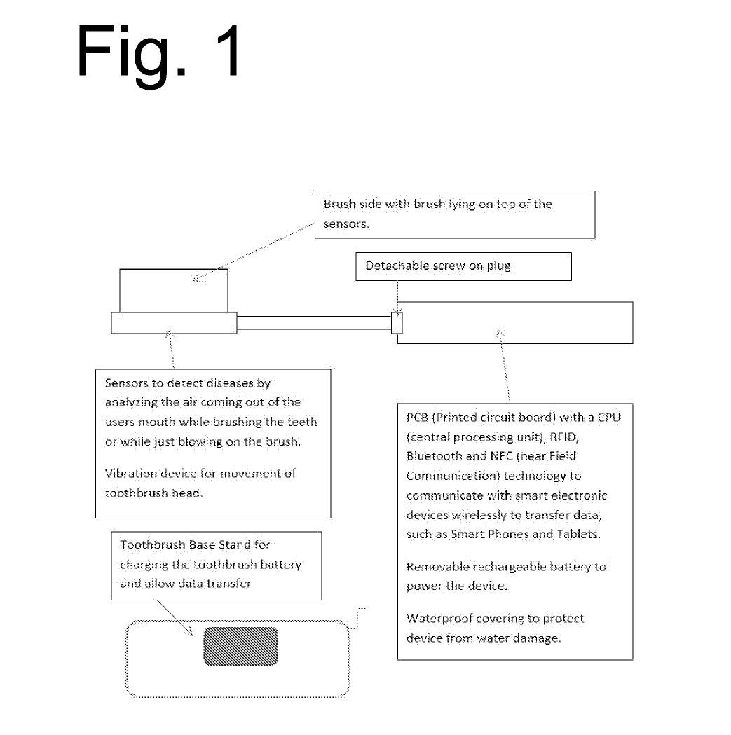

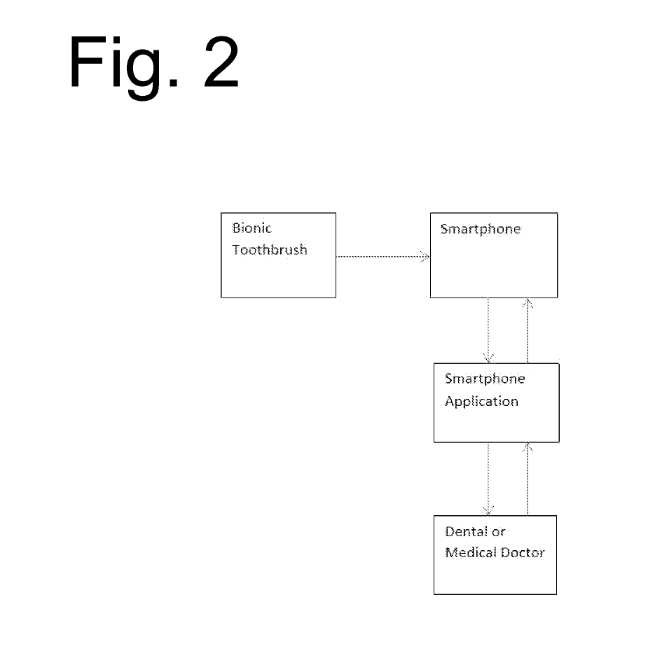

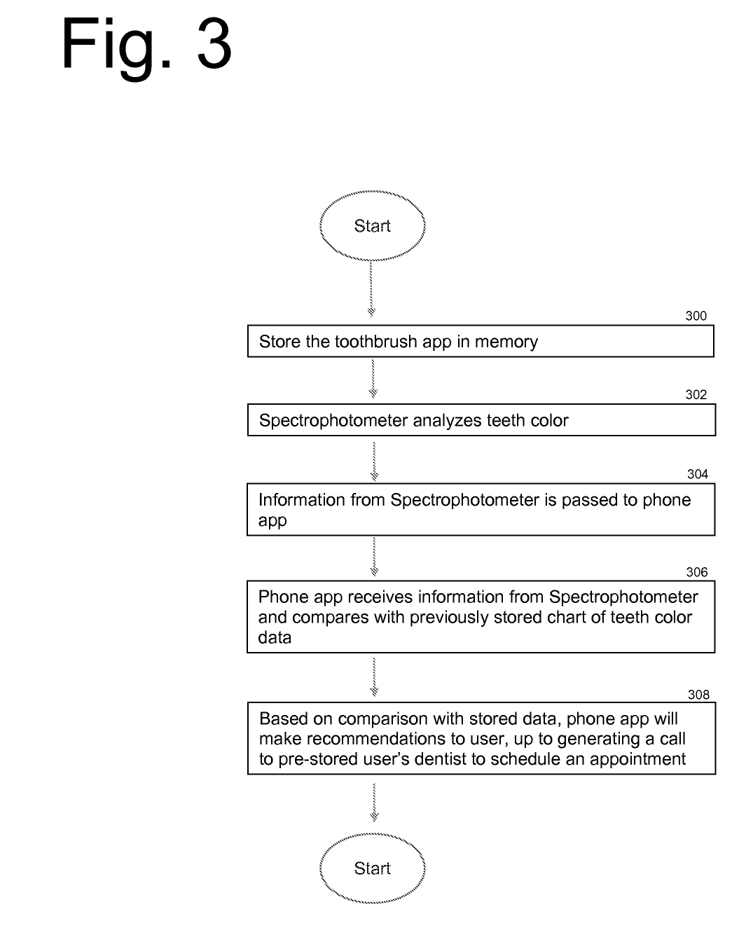

### CROSS-REFERENCE TO RELATED APPLICATIONS

Not Applicable

### SEQUENCE LISTING OR PROGRAM

Not Applicable

### BACKGROUND

### Field

This application relates to smart toothbrushes.

### BACKGROUND OF THE INVENTION

This invention relates to the field of dentistry and medicine. Many people suffer from several types of periodontal disease and other diseases that can be detected via a mouth examination, periodontal is defined as the area around the tooth, which includes the gum, periodontal membrane and bone tissue that holds the tooth in place, if a problem happens and periodontal disease is detected dental treatment will be performed by a qualified dentist and after the surgery, procedure or cleaning is performed maintenance of the proper oral hygiene is a must.

The average person only brushes the most visible areas of the tooth leaving other areas without the proper attention and without the necessary cleaning that will help avoid future dental problems and diseases. Most dentist know and recommend the Bass technique, which is considered the most efficient method for brushing the teeth.

Typical electrical toothbrushes concentrate on the proper brushing technique making sure the brush is able to access deep areas of the mouth and giving enough contact surface between the toothbrush and tooth in order to provide the necessary cleaning that will maintain a good mouth hygiene.

Conventional electrical toothbrushes only work during the time of the cleaning no data is recorded for future analysis and evaluation of the current status of the mouth, gum, tooth, oral tract, and inside gases present in the mouth. Conventional electrical toothbrushes focus their design and performance on the contact of the toothbrush bristles against the surface of the teeth, how much vibration is placed on the unit while performing the brushing of the teeth. The current state of the art, allow for basic timers that will inform the toothbrush user how long he or she needs to continue brushing the teeth, and most recent development can perform basic plaque detection.

A conventional electrical toothbrush has electrical indicators LED that will inform the user how much electrical charge are still present inside the battery, and when is necessary to recharge the rechargeable battery of the electric toothbrush. Another operation characteristic of the conventional electrical toothbrushes is presence of an ON/OFF button that allow the operator to turn the device on the cleaning operation mode, and if the user decides to turn the device off.

Another characteristics of the conventional electric toothbrushes is the presence of a wired or wireless charging base where the electric toothbrush can be placed after use, and this base placed on the top of the bathroom counter will recharge the internal rechargeable battery of the conventional electric toothbrush, no additional functionality is currently present that will make this toothbrush smart.

### SUMMARY OF INVENTION

The present invention contrives to solve the disadvantages of the prior art.

Toothbrushes are used commonly by people of all ages throughout the world for keeping proper dental hygiene. This smart toothbrush device will have an electronic board with sensors and chipsets to communicate with smart electronic devices that can do the following:

Recording of duration time of toothbrush use by the user and transferring of data to smart electronic devices.

Sensors to detect diseases from breath, saliva, oral cavity tissues of the person and also to detect temperature and pressure and transferring of the data to smart electronic devices.

The toothbrush device CPU includes an internal operating system that communicates with smart phones, tablets or computers wirelessly via Bluetooth, RFID and NFC technology. The Smartphones and Tablets or Computers need to have Bluetooth, RFID and NFC readers and an installed application in order to receive the communication. The Application installed in the smart electronic devices has not on users records of usage information, but also educational material to teach users on how to use the technologies including all the electronics and also on how to keep up with good oral hygiene. In case of detection of diseases by the sensors, the device will recommend the user to go to the doctor. The device application will use heavy security measures and encryption to prevent the users private information from transferring to any party. The user however can upon personal decision show the private information to their respective doctors when they choose to.

This device will allow users to also be able to link the devices of their kids to their smartphone, tablet or computers application to monitor or manage their kids oral hygiene. Dentists or Orthodontists or Medical Doctors will be able to use this device as a tool for giving better medical advice to their patients under their care when they and the patients have consent and are under agreement with each other.

Doctors need to download, verify their healthcare provider license and subscribe for the application in order to have an agreement to review their patients data. The subscription model adds to the revenue stream of the application. The doctors will be updated with any new upcoming updates with the technology for providing it to their patients.

Different Version of Devices:

The Bionic Toothbrush will be made in multiple versions from as simple as a device to monitor brushing the teeth habits to advanced medical sensors to detect diseases.

Data Transfer Flow Chart

User Data Transfer wirelessly from data collected and analyzed by the sensors in the Bionic Toothbrush to Smartphone device. User Data Results showcased on Smartphone device requesting them to check with doctor is sensor detected anything that's not normal. User can transfer data to their dentist or doctor if they choose to for doctor to analyze the data on the application specifically made for the users health care facilities to analyze the results and send information back to user.

Hardware Specifications:

Processor 1 GHZ or higher;

RAM 512 or higher;

Hard Drive 8 GB or higher;

Bluetooth, WiFi, RFID and NFC or something better;

Rechargeable Battery 500 MAH or something better;

Mini USB for Data Transfer or Software updates or something better;

Fingerprint Scanner or something better;

Camera: Micro camera to take photos of inside of the mouth;

Led light: White light on head of the Bionic Toothbrush;

Spectrophotometer: to detect teeth color and to use it for shade matching in the application;

Cavities detection sensors;

Colorimetric sensor to detect diseases like tumors and cancers;

Sensor to detect bad breath;

Nano-fiber sensors using Titanium DiOxide (TiO₂) or Tin DiOxide (SnO₂) nanotubes technology for a real time detection of diseases through breath

Sensors: Heart Rate Monitor, Blood Alcohol Detection Sensor, Breath Analyzer Sensors to detect various diseases and sensors to detect diseases and organism through a person's saliva and oral cavity tissues

Detachable Sensors: Blood Glucose Monitor (MEDICINE)

GRAVITRON SENSOR (ADD)

Sensor and alarm system to inform user to clean or replace the toothbrush head

Speakers embedded in device

Our toothbrush will follow current state of art, as in U.S. Pat. No. 5,894,620, where means for detecting plaque were included with an electric toothbrush, as the toothbrush cited on U.S. Pat. No. 5,894,620, only detects the presence of plaque while our invention incorporates several additional sensors that will detect much more than plaque inside the oral cavity.

In view of U.S. Pat. No. 8,585,411 where monitoring device means were provided for detecting errors in the tooth brushing process which includes a pattern reference data that was loaded inside the toothbrush including a comparator engine and an accelerometer sensor, our invention improves on the existing state-of-art by adding additional functionality not previously covered by any known invention.

Regarding the colorimetric sensor in view of U.S. Pat. No. 8,390,700, where an imaging apparatus using a colorimetric sensor is described with a light source, a colorimetric unit, and the processing unit capable to acquire colorimetric data and process the data for storage and future analysis but a medical or dental physician, were the contents of this patent is incorporated here by reference.

In WO1997014357, a heart rate monitor is described with a sensor that uses photo-optical device to detect signals corresponding to a heartbeat from periodic pulsating blood flow in a skin region by a user.

In Patent Application US20130091934, a blood collection module for measuring alcohol concentration where a blood collection module includes a blood collection container that is coupled with a blood alcohol concentration detection device including an alcohol detection sensor having a detection probe that inhales an alcohol gas, and on the surface which detect the presence of alcohol, our invention takes the existing state of the art and improves by combining this sensor and others into a much more complete method and apparatus.

In Patent Application EP2664918 A1, a breath analyzer and detachable alcohol sensor module is described, a breath analyzer which included an analyzer body and an alcohol sensor module detachably attached to analyzer body, which includes a signal processing unit for calculating an alcohol concentration.

What we claim is:

1. A method for using a smart electrical toothbrush comprising all qualities of a conventional electrical toothbrush and an electronic board with sensors and chipsets to communicate with smart electronic devices;

2. The method from claim 1, wherein the toothbrush is capable of recording of time of using toothbrush by the user;

3. The method from claim 1, wherein the toothbrush is capable of transferring of data to smart electronic devices;

4. The method from claim 1, wherein the toothbrush has sensors to detect diseases from the breath;

5. The method from claim 1, wherein the toothbrush has sensors to detect diseases from the saliva;

6. The method from claim 1, wherein the toothbrush has sensors to detect diseases from the oral cavity tissues;

7. The method from claim 1, wherein the toothbrush has sensors to detect temperature;

8. The method from claim 1, wherein the toothbrush has sensors to detect pressure;

9. The method from claim 1, wherein the toothbrush has CPU includes an internal operating system that communicates with smart phones, tablets or computers wirelessly via Bluetooth, RFID and NFC technology;

10. A method for a computer application that will received all the data from the smart electrical toothbrush, that will need to have access to the incoming data transmitted wirelessly via Bluetooth, RFID and NFC technology;

11. The method from claim 10, wherein the application installed in the smart electronic devices has not on user's records of usage information;

12. The method from claim 10, wherein the application has educational material to teach users on how to use the technologies including all the electronics and also on how to keep up with good oral hygiene;

13. The method from claim 10, wherein the application in case of detection of diseases by the sensors, the device will recommend the user to go to the doctor;

14. The method from claim 10, wherein the application has heavy security measures and encryption to prevent the users private information from transferring to any party;

15. The method from claim 10, wherein the application is capable of linking with their kids to their smartphone, tablet or computers application to monitor or manage their kids oral hygiene;

16. Means for using a smart electrical toothbrush comprising all qualities of a conventional electrical toothbrush and an electronic board with sensors and chipsets to communicate with smart electronic devices, and making the information available from the sensors for medical and dentistry analyses;

17. An apparatus for using a smart electrical toothbrush comprising all qualities of a conventional electrical toothbrush and an electronic board with sensors and chipsets to communicate with smart electronic devices
